(19) United States
(12) Patent Application Publication
(10) Pub. No.: US 2004/0219508 A1
(43) Pub. Date: Nov. 4, 2004

(54) Screening, quantitation and identification of active ingredients in natural products

(75) Inventor: Gary K. Schwartz, Briarcliff Manor, NY (US)

Correspondence Address: Albert Wai-Kit Chan, Law Offices of Albert Wai-Kit Chan, LLC, World Plaza, Suite 604, 141-07 20th Avenue, Whitestone, NY, 11357, US

(73) Assignee: Sloan-Kettering Institute for Cancer Research

(21) Appl. No.: 10/693,301

(22) Filed: Oct. 24, 2003

Related U.S. Application Data
(60) Provisional application No. 60/421,411, filed on Oct. 25, 2002.

(51) Int. Cl. C12Q 1/00

(52) U.S. Cl. 435/4

(57) ABSTRACT

This invention provides a method for screening a mixture of compounds for activity comprising steps of contacting the mixture with a system which mimics an organ capable of metabolizing the mixture in an appropriate time to generate a metabolite; and determining the activity of the generated metabolite. This invention also provides a method for quantitating a mixture of compounds comprising steps of contacting the mixture with a system which mimics an organ capable of metabolizing the mixture in an appropriate time to generate a metabolite; and determining the activity of the generated metabolite. This invention further provides a method for identifying an active metabolite from a mixture of compounds comprising steps of contacting the mixture with a system which mimics an organ capable of metabolizing the mixture in an appropriate time to generate a metabolite; and determining the activity of the metabolite generated. Finally, this invention provides various uses of this method and the identified metabolite.

This application claims priority of U.S. Ser. No. 60/421,411, filed 25 Oct. 2002, the content of which is incorporated by reference here into this application.

The invention disclosed herein was made with United States Government support under U.S. Army DOD Breast Cancer Grant No. DAMD17-02-0486 from the United States Department of Department of Defense. Accordingly, the United States Government has certain rights in this invention.

Throughout this application, various publications are referenced. Disclosures of these publications in their entireties are hereby incorporated by reference into this application in order to more fully describe the state of the art to which this invention pertains.

### BACKGROUND OF THE INVENTION

Herbal medicine has been used for centuries in the world. It is still an important component in the healthcare system, including prevention and treatment of diseases, particularly in Asian countries. Until recently, most pharmaceuticals were originated from natural products; flowering plants and ferns have produced about 120 commercial drugs and account for 25% of all prescriptions issued in North America every year. For example, reserpin, ephedrine, digitalis, morphine, vincristine, taxol, harringtonin, just name a few. In many laboratories over the world, investigators use advanced technology to analyze the properties of herbal medicines to search for active ingredient. Identification and isolation of the active ingredient from herbal medicine is one of the ways to discover useful therapeutic agent.

Herbal medicine, for example, traditional Chinese medicine (TCM) has been used as multiple formulae for thousands of years. There is abundant information on the safety and efficacy of these formulae for treating various diseases. However, because of the limitation of the technology and knowledge, there is no effective system to evaluate the quality, purity, stability, and consistency of herbal medicine, which makes evaluation of the clinical efficacy difficult. The trend of disease management, at least for cancer and viral disease, has changed to monotherapy to drug combinations. For example, drugs such as alkylating agents, hormones, anti-emetics, anti-tumor antibiotics may be included as a regimen for the treatment of many cancers.

TCM has been used as combinations, many of them serve different function, such as decrease drug metabolism or toxicity, or increase absorption, solubility, or increase efficacy by acting on different mechanisms. Using a mixture of plant extract instead of using an isolated compound for the management diseases has gained acceptance in Western countries. The difficulties for validating the efficacy or the utility of herbal medicine is lack of a quality control system to guarantee the quality of the pharmaceutical preparation for clinical trials.

Chromatographic and spectroscopic methods and other physical chemical methods such as high performance liquid chromatograph, gas chromatograph, mass spectrometer, nuclear magnetic resonance spectrometer, UV/visible spectrophotometer, and melting point have been used to characterize the active ingredient of the pharmaceutical preparation. These techniques are extremely useful to set specification for the drug substance and finish product because the structure and identity of the active component is known. It is technically difficult if not impossible to use the above techniques to set specification for the drug substance or finish product for herbal medicines. Without knowing the consistency of the manufacturing process and the quality of the drug, it is impossible to conduct clinical trials to evaluate the efficacy of the drug.

Herbal medications are currently being promoted for clinical use in cancer therapy. However, many of the claims made for these herbal remedies are based on anecdotes in traditional Chinese medicine and, unfortunately, are not grounded in scientific fact. Nevertheless, many of the chemotherapeutic agents that are in clinical use today are derived from plants and natural products. For example, paclitaxel is a plant product from the stem bark of Taxus brevis, the western yew. Therefore, the possibility that one or more of these claims is true can not be discounted. The challenge is how to prove that one of these claims may have scientific validity and how to test this within the confines of Western medicine tradition.

In this invention, Huanglian (coptis chinesis) is used as an example to prove the concept. Huanglian is an herbal tea that has been widely used in China for several thousand years. It is prepared as an herbal tea from the roots of Rhizoma coptidis. In traditional Chinese medicine it has been used to treat inflammatory conditions ranging from gastroenteritis to acute febrile illnesses with no reported toxicity.

Huanglian has been reported in vitro to inhibit the growth of heliobacter pylori and the intestinal parasite blastocystis hominis (1-3). It inhibits lipid peroxidation in rat tissues and protected rats from chemically induced diabetes (4), possibly a result of free-radical removal. The serum of rats which had received oral coptis chinesis inhibited the biotransformation of arachidonic acid(5).

Huanglian studies have explored its usefulness as a component of a salve to treat burn wounds (6), as a component of a herbal mixture to inhibit platelet aggregation (7).

Extracts of huanglian have been shown to inhibit topoisomerase I to levels that are equivalent to that of camptothecins used in clinical oncology today (8,9). Huanglian in fact is a complex mixture of compounds. The largest component of huanglian has been identified as berberine but other constituents including coptisine, palmatine, jatrorrhizine, bauerenol, and epiberberine have been identified (10). Continuous exposure of HepG2 hepatoma cells to berberine (1 to 50 μM) inhibits tumor cell growth in a dose-dependent manner 11. This was associated with a decrease in both the S phase fraction of the cells and in the secretion of alpha-fetoprotein. Oral administration of huanglian to laboratory rats inhibits the formation of azoxymethane (AOM)-induced aberrant crypt foci, a putative pre- neoplastic lesion for colon cancer (12). Huanglian has been administered to patients as an aqueous, herbal tea at doses of 20 to 30 grams/day without toxicity. Its role as an anti-cancer drug has not been defined. It is not known which are the active components. In fact, the large range of components within huanglian may result in a broad-based inhibition of many cancer targets, including topoisomerase I. This demonstration of anti-cancer effects in vitro and identification of new targets provide a rationale for clinical development of this agent as a whole herb in cancer therapy.

Huanglian administration to mice at a dose of 27 g/kg/d produced no toxicity(13). In newborn infants, huanglian has been shown to increase unconjugated bilirubin in newborns presumably by displacing bilirubin from serum binding protein. The use of this herb is advised against in neonates in Southern China, where neonatal hyperbilirubinemia is prevalent(14). Bilirubin displacement from albumin may be due to the huanglian component berberine(15).

A number of huanglian components are known, some have been tested as purified compounds for a variety of clinical conditions. Berberine, an alkaloid of the protoberberine family, represents the most abundant (50%) component of huanglian extract. In dogs berberine has been shown to have positive inotropic effects and lower peripheral vascular resistance. In 12 patients with refractory heart failure berberine was given intravenously at 0.02 mg/kg/min over 30 minutes, and at 0.2 mg/kg/min over 30 minutes. At the higher dose, a decrease in pulmonary and systemic vascular resistance and an increase in cardiac index and ejection fraction were seen. Four of 12 patients, however, experienced ventricular tachycardia with “torsades de pointes” morphology within 1-20 hours of the infusion. Cardiac diagnoses in these patients were: 2 patients with idiopathic dilated cardiomyopathy, one with ischemic heart disease, and one with Chaga's disease. The investigators noted that remarkable hemodynamic stability was observed during the VT episodes. The QTc interval was consistently elevated in all 12 patients after berberine, and returned to baseline after 24 hours after drug administration. Other side effects were facial flushing (2 patients) and transient nausea (2 patients)(16).

Zeng and Zeng treated 56 patients with chronic heart failure (CHF) with berberine by mouth, 1.2 gm/d for two weeks. Patients with higher berberine plasma concentrations (>0.11 mg/L) experienced a more significant increase in LVEF and decrease in the frequency and complexity of ventricular premature beats (VPBs). When given in this manner to patients with CHF no side effects or arrythmogenesis was reported(17).

This invention discloses results that huanglian potently inhibits the growth of cancer, e.g.: gastric, breast, and colon cancer cells in vitro in a dose-dependent manner. There is maximal inhibition at low micromolar concentrations. In addition this degree of inhibition is associated with suppression of cyclin B1 protein and cyclin dependent kinase 1 (cdc2 kinase) activity. This invention discloses that huanglian represents a novel cancer drug that mediates suppression of cancer growth in association with down-regulation of cyclin B1.

### DETAILED DESCRIPTION OF THE FIGURES

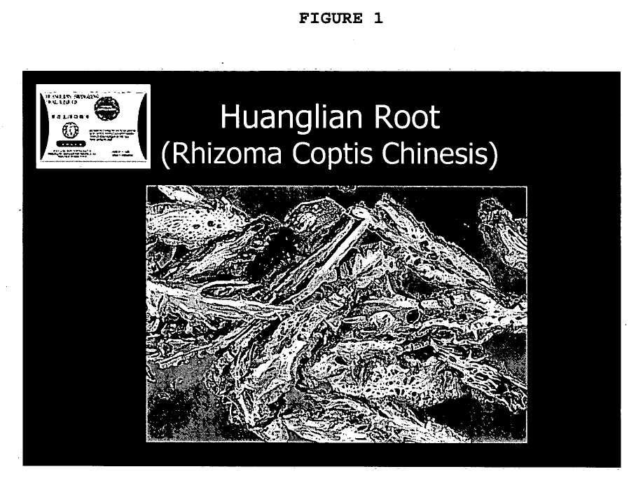

FIG. 1. Huanglian Root (Rhizoma Coptis Chinesis)

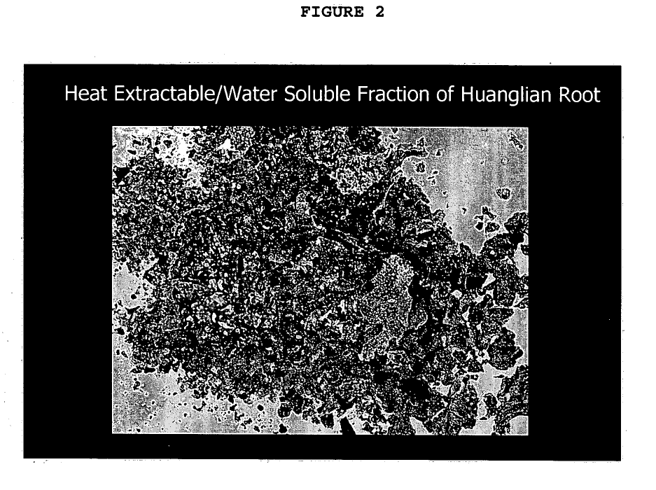

FIG. 2. Heat Extractable/Water Soluble Fraction of Huanglian Root

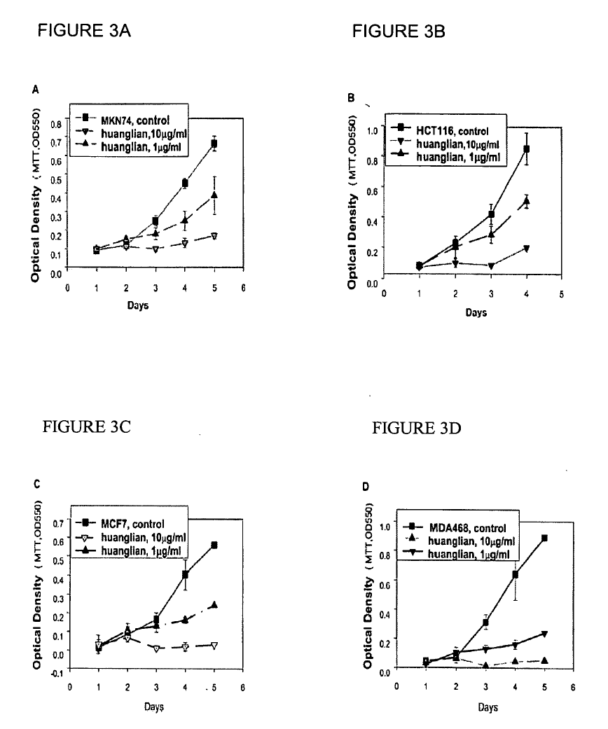

FIG. 3. Huanglian Inhibits Growth of Human Tumor Cell Lines in Vitro

FIG. 3A. Gastric MKN-74

FIG. 3B. Colon: HCT-116

FIG. 3C. Breast: MCF-7(p53⁺)

FIG. 3D. Breast: MDA468(p53⁻)

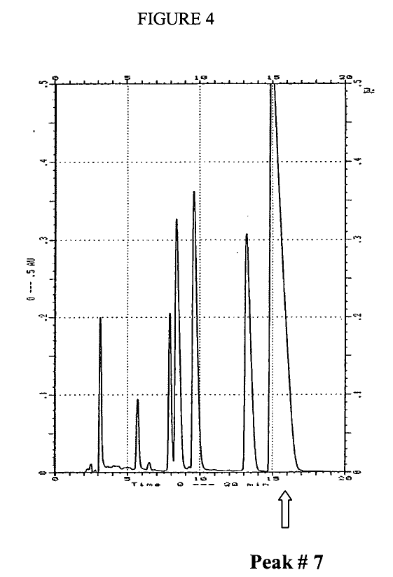

FIG. 4. Huanglian: 7 Peaks by HPLC with Berberine (peak #7) Constituting 50% of the Water/Heat Extractable Product

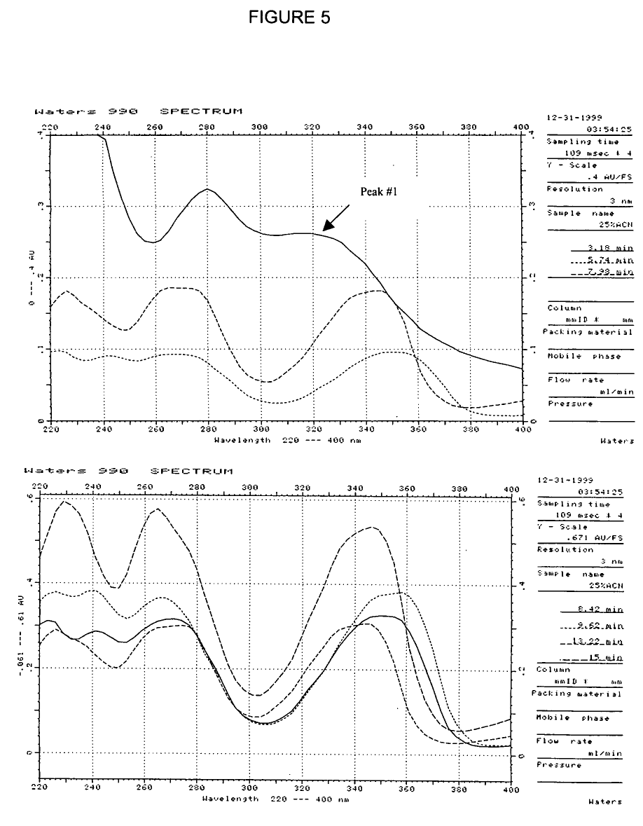

FIG. 5. UV Spectra Indicate All Peaks, but Peak 1, are Berberine or “Berberine-like”

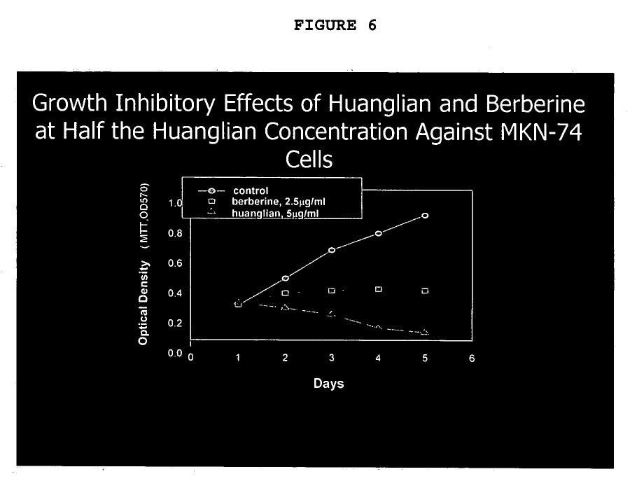

FIG. 6. Growth Inhibitory Effects of Huanglian and Berberine at Half the Huanglian Concentration Against MKN-74 Cells

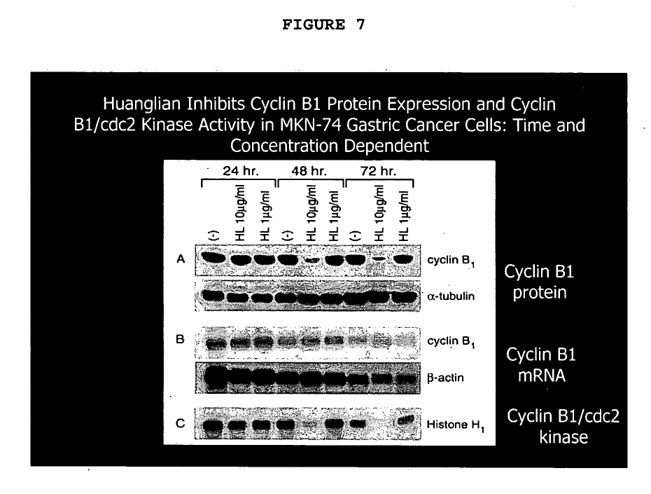

FIG. 7. Huanglian Inhibits Cyclin B1 Protein Expression and Cyclin B1/cdc2 Kinase Activity in MKN-74 Gastric Caner Cells: Time and Concentration Dependent

FIG. 7A. Cyclin B1 protein

FIG. 7B. Cyclin B1 mRNA

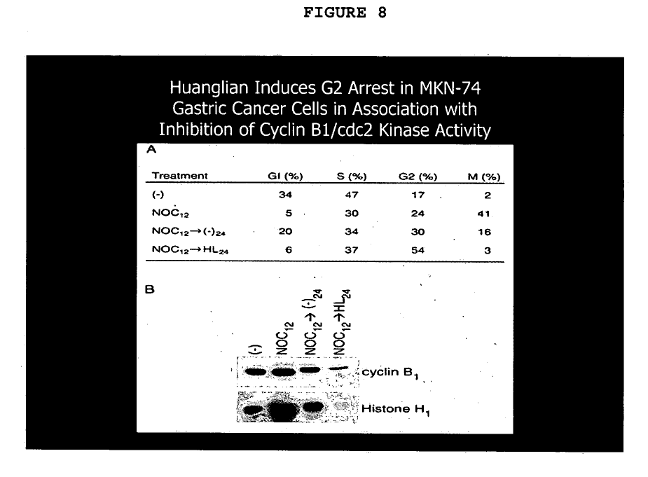

FIG. 8. Huanglian Induces G2 Arrest in MKN-74 Gastric Cancer Cells in Association with Inhibition of Cyclin B1/cdc2 Kinase Activity

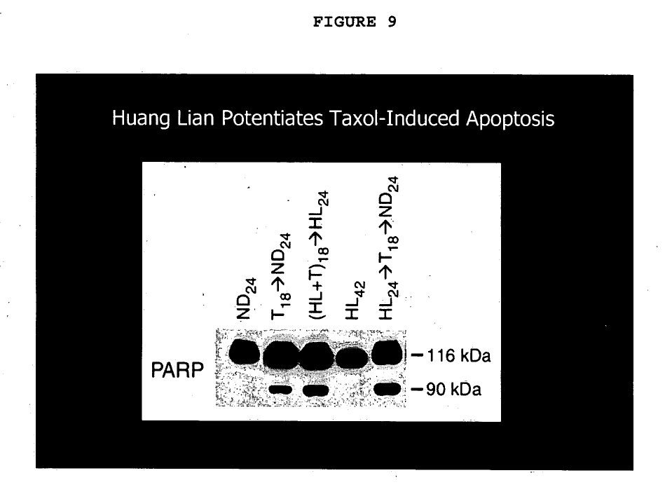

FIG. 9. Huang Lian Potentiates Taxol-Induced Apoptosis

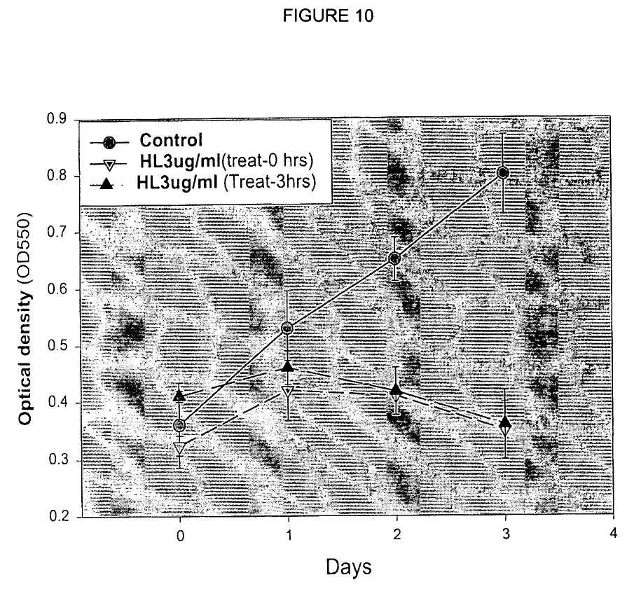

FIG. 10. Effect of Huanglian Metabolites Formed by Human Liver S9 Microsomes on MKN-74 Gastric Cancer Cell Growth: First Pass Effect

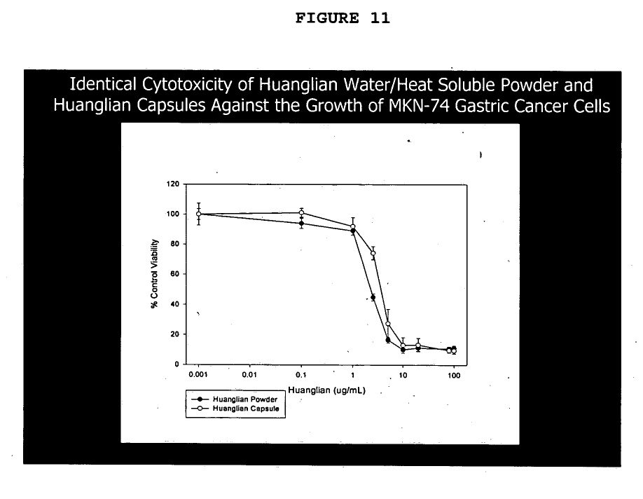

FIG. 11. Identical Cytotoxicity of Huanglian Water/Heat Soluble Powder and Huanglian Capsules Against the Growth of MKN-74 Gastric Cancer Cells

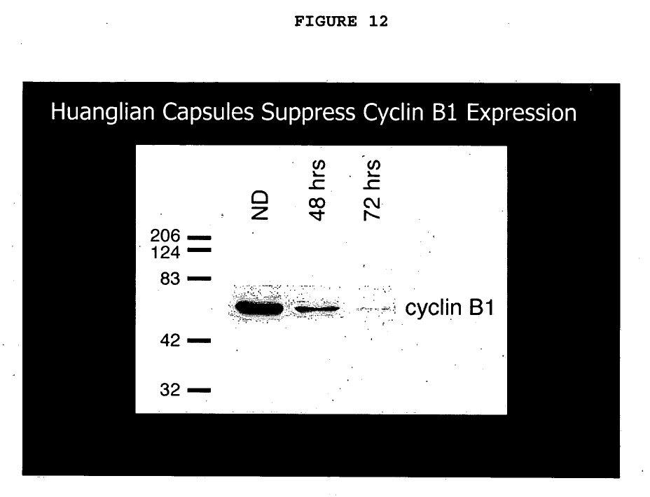

FIG. 12. Huanglian Capsules Suppress Cyclin B1 Expression

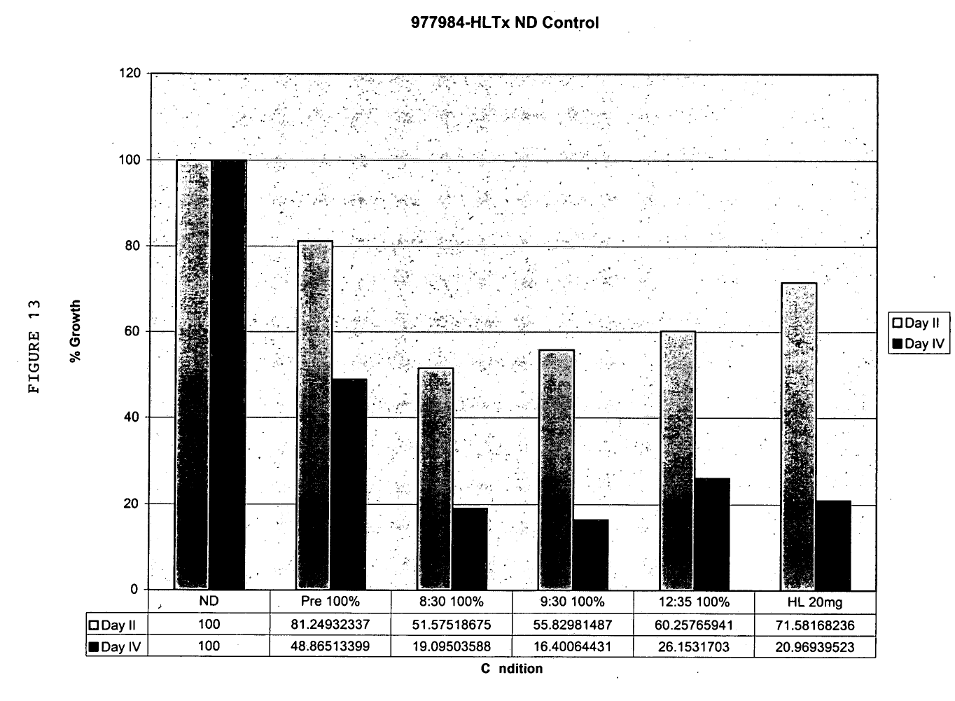

FIG. 13. Plate 5000 MKN-74 cells in 200 microliters of media (minimum essential media (MEM)+10% FBS) in each well of a 96-well plate. The cells are allowed to grow for 24 hours. Meanwhile both pretreatment and post-treatment plasma (day #15) are split into 3 one ml samples and concentrated in a Centrivap Concentrator (LAB CONCO) for approximately 5½ M hours at 35 degrees C. After the concentration, the final 300 microliter of concentrated plasma is reconstituted back to 1 mL by adding 700 microliter of media (MEM+10% FBS). Then the three 1 ml samples are then pooled to make a final 3 mL volume to be used for baseline and for post treatment testing. For the positive control 6 microliters of stock huanglian (10 mg/mL) are added to 3 ml of medium 20 (MEM+10% FBS) to yield 20 micrograms/mL final concentration.

Take the 96-well plates after 24 hours and gently remove the original media. Add the following treatments (200 microliters) in triplicate to each well of the plate: media alone, huanglian in media (positive control), pretreatment plasma (pre), and post-treatment from predetermined times on day #15 (example 8:30 AM, (:30 AM, and 12:35 PM). Cells are treated for 2 (day II) or 4 (day IV) days and then tested for cell survival by the SRB assay.

For the SRB assay we follow the published SRB protocol (sulforhodamine B). Remove the media, wash 3× with 100 microliter of phosphate buffer soda (PBS), then add 10% cold trichloacetic acid (TCA) for 30 minutes at 4 degrees centigrade. Remove the TCA, wash with water, invert plate and gently tap out excess water. Add 25 to 30 microliters of SRB in 1% acetic acid to each well. Incubate for 5 minutes at room temperature. Repeat ×4. Then add 100 microliters of 10 mM Tris buffer and put on shaker for 10 minutes. After agitation, wipe plates with alcohol and then run plate in spectrophotometer (Spectromax—340 PC, Molecular Devices) and read at 490 nm. Proliferation correlates to increase in red color.

As shown for patient 977984 her pretreatment plasma decrease cell growth from 100% to 82% with 2 days of plasma exposure, and with 4 days of exposure this had decreased from 100% to 48% indicating that the patients own plasma (even before treatment) can decrease cell proliferation of the MKN-74 cells. However, using the day #15 plasma samples for 3 respective time points (8:30, 9:30, and 12:35) there is an additional inhibition in cell growth. As shown, for these 3 time points there is 50 to 60% inhibition in cell growth for the 2 days of plasma exposure (compared to 80% with the pretreatment plasma with 2 days of plasma exposure). This then decrease further for all time points to approximately 80% inhibition with 4 days of plasma exposure (compared to 48% with the pretreatment plasma after 4 days of plasma exposure). Three time points show excellent correlation with a very small standard deviation. This confirms that the assay is working, as 20 micrograms/ml inhibits cell proliferation of MKN-74 cells by 80% with 4 days of drug exposure.

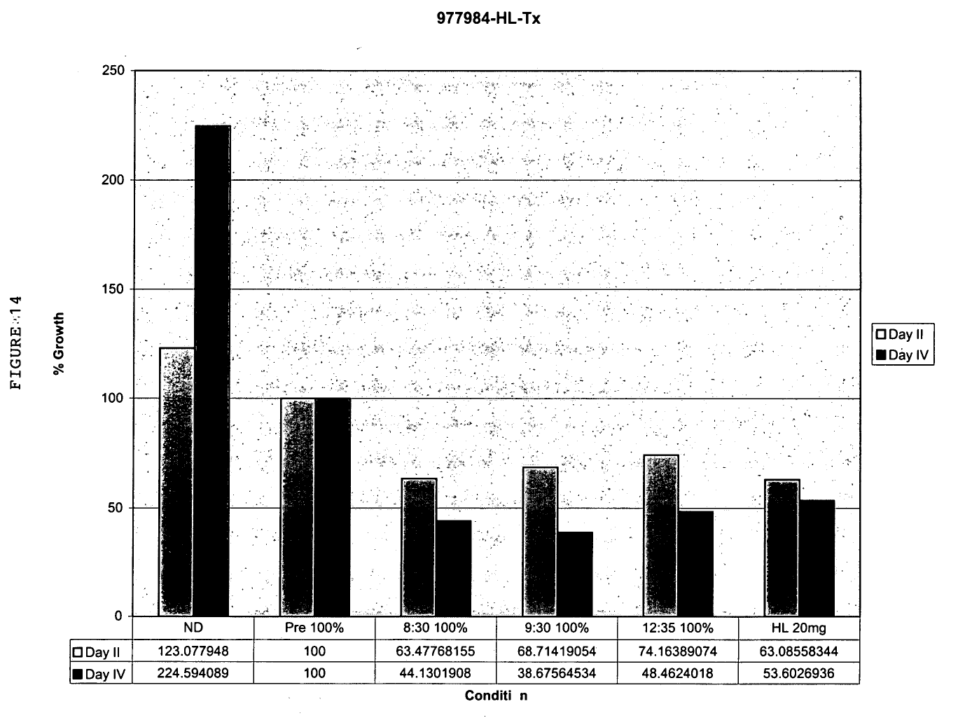

FIG. 14. Same data as presented in FIG. 13, normalized with pretreatment plasma.

### DETAILED DESCRIPTION OF THE INVENTION

This invention provides a method for screening a mixture of compounds for activity comprising steps of contacting the mixture with a system which mimics an organ capable of metabolizing the mixture in an appropriate time to generate a metabolite; and determining the activity of the generated metabolite.

This invention provides a method for quantitating a mixture of compounds comprising steps of contacting the mixture with a system which mimics an organ capable of metabolizing the mixture in an appropriate time to generate a metabolite; and determining the activity of the generated metabolite.

This invention provides a method for identifying an active metabolite from a mixture of compounds comprising steps of contacting the mixture with a system which mimics an organ capable of metabolizing the mixture in an appropriate time to generate a metabolite; and determining the activity of the metabolite generated.

As used herein, the mixture of compounds may be obtained from many sources. In an embodiment, the mixture is from a natural product. In another embodiment, the natural product is an herbal medicine.

“Herbal medicine,” for the purpose of this specification means that herbs or natural products with therapeutic values, whose active ingredients are known or unknown, are processed by extraction and/or concentration that mixed with pharmaceutical carriers that are suitable for therapeutic uses. Examples for herbal medicine includes but not limited to extraction with water and/or alcohol and/or mineral oil with different ratios with or without heating. Extracts may or may not be further processed into various dosage forms such as capsule, piles, or concoctions. In accordance with the present invention, herbal medicine is prepared according to any of the known procedures that are suitable for therapeutics.

As used in this invention, there are known systems which mimic an organ capable of metabolizing the mixture in an appropriate time to generate a metabolite. In an embodiment, the organ is a liver. In a further embodiment, the system is human liver microsomes.

This invention provides a method for screening a mixture of compounds for activity comprising steps of administering the mixture to a subject capable of metabolizing the mixture; and taking bodily fluid that contains a metabolite of the mixture from the subject to determine the activity of the metabolite generated.

As used herein, “bodily fluids” may include but not be limited to blood, urine and lymphatic fluid.

This invention provides a method for quantitating an herbal extract comprising steps of administering the mixture to a subject capable of metabolizing the mixture; and taking bodily fluid that contains a metabolite of the mixture from the subject to determine the activity of the metabolite generated.

This invention provides a method for identifying an active metabolite from a mixture of compounds comprising steps of administering the mixture to a subject capable of metabolizing the mixture; and taking bodily fluid that contains a metabolite of the mixture from the subject to determine the activity of the metabolite generated.

The activity of the metabolite may be determined by in vitro assay. In an embodiment, the assay is determined by the inhibition of cell growth. In a further embodiment, the cells are cancerous cells.

In accordance with the present invention, the activity of the metabolite is determined by the inhibition of cyclin B1 activity. In an embodiment, the inhibition of cyclin B1 activity is at least 50%.

This invention provides the active metabolite identified by the above method. This invention also provides a composition comprising the metabolite and a carrier.

This invention provides a pharmaceutical composition comprising an effective amount of the identified metabolite and a pharmaceutically acceptable carrier.

For the purposes of this invention, “pharmaceutically acceptable carriers” means any of the standard pharmaceutical carriers. Examples of suitable carriers are well known in the art and may include, but not limited to, any of the standard pharmaceutical carriers such as a phosphate buffered saline solutions, phosphate buffered saline containing Polysorb 80, water, emulsions such as oil/water emulsion, and various type of wetting agents. Other carriers may also include sterile solutions, tablets, coated tablets, and capsules.

Typically such carriers contain excipients such as starch, milk, sugar, certain types of clay, gelatin, stearic acid or salts thereof, magnesium or calcium stearate, talc, vegetable fats or oils, gums, glycols, or other known excipients. Such carriers may also include flavor and color additives or other ingredients. Compositions comprising such carriers are formulated by well known conventional methods.

This invention provides a method of producing a fingerprint or profile thereof of an extract of a natural product comprising steps of contacting the extract with a system which mimics an organ capable of metabolizing the extract in an appropriate time to generate a metabolite; and determining the identity and amount of the metabolite generated, thereby generating a fingerprint of the extract. In an embodiment, the organ is a liver. In a further embodiment, the system is human liver microsomes.

This invention provides a method of producing a fingerprint or profile thereof of an extract of a natural product comprising steps of administering the extract to a subject capable of metabolizing the extract; and determining the metabolite generated, thereby generalizing a fingerprint of the extract. In an embodiment, the natural product is an herb.

This invention provides the produced fingerprints and uses thereof.

This invention provides a method to determine the batch-to-batch variation of an extract from a natural product comprising comparison of the characteristics of the produced fingerprints of different batches.

This invention provides a method to assay for the formulation variation of an extract from a natural product comprising comparison of the characteristics of the produced fingerprints of different batches.

This invention provides a method to assay for the dose variation of an extract from a natural product comprising comparison of the characteristics of the produced fingerprints of different batches.

The fingerprint or profile presented may provide a method to set specification for pharmaceutical grade of herbal medicines. The fingerprint or profile presented may be used to standardize the manufacturing process for producing pharmaceutical grade of herbal medicine. For example, by determining the profile of different lots of herbal medicine, one can identify the most reproducible manufacturing process. This invention also provides a quality control method to assure the quality of the pharmaceutical preparation which is important to guarantee the efficacy and the safety profile of the pharmaceutical preparation. Pharmaceutical preparations consisting of similar chemical ingredients will exhibit a similar profile.

This invention provides a method for identifying induced compounds in a subject comprising steps of administering a mixture of compounds to the subject; extracting bodily fluid from the subject to determine the generation of induced compounds; and identifying the said induced compounds. In an embodiment, the mixture is an extract from a natural product.

This invention also provides the induced compounds identified by the above method. In an embodiment, the subject is a human.

This invention provides a method for treating cancer in a subject comprising administering to the subject an effective amount of coptis chinesis extract. The cancer includes, but is not limited to, renal, colon, sarcoma, neuro, lung, breast, prostate, stomach, esophageal, pancreatic, bladder, lymphoma, leukemia, and hepatoma. In another embodiment, the cancer is a solid tumor.

This invention provides a method for treating cancer in a subject comprising administering to the subject an effective amount of coptis chinesis extract and a therapeutic agent.

As used herein, the therapeutic agent is any of the known agents capable of providing therapeutic effects to a subject. The therapeutic agent includes but is not limited to irinotecan, gemcitabine, doxorubicin, and cisplatin. A list of therapeutic agents can be found in Cancer Principles and Practice of Oncology, 5th Edition, 1997, Edited by Vincent T. DeVita, Samuel Hellman and Steven A. Rosenberg, Published by Lippincott Williams & Wilkens Publishers. In an embodiment, the therapeutic agent is a microtubule-destabilizing agent. In a further embodiment, the microtubule-destabilizing agent is a taxol or a taxol-like compound.

U.S. Pat. No. 6,444,638 discloses that an antitumor therapeutic agent in combination with a modulating agent is capable of increasing apoptosis in tumor cells. In an embodiment; the modulating agent is a protein kinase C inhibitor. The modulating agent includes, but is not limited to Safingol (L-threo-dihydrosphingosine), Ro-1 (Bisindolylmaleimide), Ro32-0432 (Bisindolylmaleimide tertiary amine), UCN-01 (7-OH-staurosporine), Flavopiridol (L-86-8275), Bryostatin 1 (macrocyclic lactone), and antisense nucleotides capable of inhibiting the expression of protein kinase C. This invention, therefore, also encompasses coptis chinesis extract, a therapeutic agent and a modulating agent.

The treatment of the coptis chinesis extract and the therapeutic agent may be performed in a sequential manner. In a further embodiment, the subject is treated with coptis chinesis extract first, then a therapeutic agent.

This invention provides an anti-tumor composition comprising an effective amount of coptis chinesis extract. This invention also provides a pharmaceutical composition comprising coptis chinesis extract.

This invention will be better understood from the Examples which follow. However, one skilled in the art will readily appreciate that the specific methods and results discussed are merely illustrative of the invention as described more fully in the claims which follow thereafter.

### EXPERIMENTAL DETAILS

### EXAMPLE 1

### Preparation of Huanglian Extract

In a boiled “tea” the only fraction that is in fact consumed is that which enters the aqueous phase. The remainder, or the “grinds,” which comprises most of the product, is in fact discarded. In traditional Chinese medicine, patients prepare up to 30 grams of huanglian for tea each day. Therefore, 30 grams of huanglian were added directly to 1 liter of distilled water. The solution was heated to 100° C. for 1 hour. Since the insoluble root fibers or “grinds” are not consumed, they were sterilely removed by filtration through 0.2 micron filter paper. In order to have huanglian suitable for drug studies, the remaining aqueous solution was boiled to dryness. This yielded approximately 1.5 grams of huanglian. It is believed this extract of huanglian is equivalent to the amount of actual huanglian tea a patient ingests each day when the huanglian is prepared as an herbal tea. The dry powder was then weighed and a stock solution of 1 mg/ml was made in water for subsequent studies.

### EXAMPLE 2

### Inhibition of Cell Growth by Huanglian and its Metabolites

FIG. 3 shows the growth curves for the human cancer cell lines MKN-74 (gastric Panel A), HCT-116 (colon, Panel B), MCF-7 (breast, Panel C) and MDA468 (breast, Panel D) following treatment with 10 and 1 μg/ml of huanglian. The cells were treated on day 0 with huanglian and then cell viability in the continued presence or absence (control) of drug was determined for the subsequent 5 days (days 1 to 5) by the MTT (3-(4,5-dimethythiazol-2-yl)-2-5-diphenyltetratrazolium bromide) assay. In this colorometric assay the absorbance of converted dye is measured at a wavelength (OD) of 550 nm and the increased absorbance is directly proportional to cell viability. As shown, huanglian induced a dose dependent inhibition of cell growth in all four cell lines. By day 3 with 10 μg/ml of huanglian, this approached 100% growth inhibition. When compared to untreated controls, the inhibition of cell growth persisted until completion of the studies on days 4 or 5. Interestingly, this effect appeared to be independent of the p53 status of the cells, as growth suppression by huanglian was similar for MCF-7, which is wild-type for p53, and MDA468, which is mutant for p53.

### EXAMPLE 3

### Inhibition of Colony Formation

These studies were then extended to test the effect of huanglian on colony formation (data not shown). For these assays the same cell lines (HCT116, MKN74, MDA468, and MCF7) were grown in 6-well plates and then exposed to huanglian (1, 10, and 100 μg/m) or drug free medium (ND) for two weeks. At the end of two weeks the colonies that formed were stained with crystal violet and counted. Colony formation was calculated as a percentage of untreated controls. These resultsu are shown below and indicate a dose-dependent suppression of colony formation with huanglian concentrations of 1, 10, and 100 μg/ml. Except in the case of HCT116 cells, there was absolutely no colony formation with 10 μg/ml huanglian.

### EXAMPLE 4

### Suppression of Cyclin B1 Protein Expression

In view of inventor's interest on cell cycle drugs as well as reported effects of berberine, the major constituent of huanglian, on cell cycle distribution, the effect of huanglian was tested on the expression of cyclins associated with the cell cycle (FIG. 7). For these studies MKN-74 gastric cancer cells were treated at time 0 with drug free medium (−) or medium containing 1 and 10 μg/ml huanglian (HL). Protein lysates were then prepared for 24, 48, or 72 hours later and transferred onto Immobilion membranes. The membranes were then probed with mouse monoclonal cyclin B1 (kindly provided by Dr. Tim Hunt, ICRF Clare Laboratories, UK) and then treated with a secondary antimouse-HRP antibody. Detection was done by ECL Chemiluminescence. As shown in the western blot in FIG. 7A, 10 μg/ml of huanglian suppressed cyclin B1 expression after 48 and 72 hours of drug exposure, when compared to untreated controls. Equal protein loading was confirmed with α-tubulin.

The decrease in cyclin B1 protein expression correlated to the inhibition of cell growth in the tumor cell lines shown in FIG. 7. Experiments were designed to determine whether the loss of cyclin B1 protein was related to a decrease in cyclin B1 mRNA expression. These results are shown in the Northern blot in FIG. 7B. Even though overall RNA content appear to decreased over time, there was no effect by huanglian on cyclin B1 mRNA expression up to concentrations of 10 μg/ml at 24, 48, or 72 hours, when compared to the untreated controls. Equal RNA loading was confirmed with β-actin controls. These results would indicate that huanglian decreases the expression of cyclin B1 at either a translational or post-translational level. In order to determine specificity of huanglian for cyclin B1, the effect of huanglian on protein expression of other cyclins has begun to be examined. Thus far, concentrations of 10 μg/ml huanglian do not suppress the protein expression of cyclin A or E after 24, 48, and 72 hours of drug exposure (data not shown).

### EXAMPLE 5

### Suppression of cdc2 Kinase Activity by Huanglian

Cyclin B1 is the cyclin that binds to and activates cyclin dependent kinase 1 (cdc2 kinase)(20). Therefore it was hypothesized that the loss of cyclin B1 protein should result in a decrease in the enzymatic activity of cdc2 kinase. For these studies the MKN-74 cells were again treated with 1 or 10 μg/ml of huanglian for 24, 48, and 72 hours. Soluble protein was immunoprecipitated with anti-cyclin B1 (Santa Cruz, Biotech. Inc, Calif.). This immunocomplex contains cyclin B1 associated cdc2 kinase. The activity of the complex was then determined by incorporation of [³²P]ATP into a histone H1 substrate. The results are shown in FIG. 7C. As predicted, the suppression of cyclin B1 protein resulted in a decrease in cdc2 kinase activity (reflected as a decrease in H1 phosphorylation) after exposure to 10 μg/ml of huanglian for 48 and 72 hours. In fact, by 72 hours cdc2 kinase activity is completely inhibited when compared to the untreated control. This effect of huanglian on inhibiting cdc2 kinase activity would be considered indirect since it is mediated by suppression of the putative upstream cdc2 activator, cyclin B1. Experiments were designed to determine whether huanglian could directly inhibit cdc2 kinase. For these studies cyclin B1 associated cdc2 kinase was immunoprecipitated from untreated cells and 10 μg/ml of huanglian was added directly to the kinase assay. Under these in vitro conditions, no direct inhibition of cdc2 kinase by huanglian was observed (data not shown), indicating that the inhibition of cdc2 kinase by huanglian must be mediated by suppression of cyclin B1.

### EXAMPLE 6

### Effect of Huanglian on G2/M Transition

Activation of cyclin B1 associated cdc2 kinase by mitotic inhibitors including paclitaxel induces an increased accumulation of cells into the M phase of the cell cycle (12). Thus, a decrease in cyclin B1 associated kinase activity should prevent cells from entering M and results in an accumulation of cells in G2. It should also force cells in M to exit M and continue to cell cycle. Experiments were designed to test this in the MKN-74 cells by flow cytometry and labeling of cells with the MPM-2 antibody which identifies cells in mitosis. This is difficult to assess in asynchronous cells. So as to better document accumulation of cell in G2, experiments were designed to first synchronise the cells with nocodazole in the M phase (e.g. mitotic block). The results are shown in FIG. 8A.

Treatment with nocodazole for 12 hours (NOC₁₂) resulted in 41% of the cells remaining in M. When cells are first treated with nocodazole and then treated with drug free medium for an additional 24 hours (NOC₁₂→(−)₂₄), the cells are released from mitosis, though 16% of the cells still remain in M. In contrast, when the nocodazole therapy is followed by 24 hours of huanglian (NOC₁₂→HL₂₄), the cells have accelerated their exit out of M and have begun to accumulate in the G2 phase of the cycle. In order to prove that this effect by huanglian on the nocodazole treated cells was in fact due to suppression of cyclin B1 and inhibition of cdc2 kinase, protein lysates were also made from these cells and tested for cyclin B1 expression by western blot and cdc2 kinase activity by histone H1 phosphorylation. As shown in FIG. 8B, under the condition of NOC₁₂→HL₂₄, there was suppression of cyclin B1 protein and cdc2 kinase activity relative to NOC₁₂→(−)₂₄. This data supports the hypothesis that huanglian suppresses cdc2 kinase activity in association with loss of cyclin B1 protein, resulting in a G2 block.

### EXAMPLE 7

### Huanglian Assessment by HPLC and Atomic Absorption

The initial analysis of huanglian was based on published methods (21, 22). After further testing, techniques were refined as follows. Huanglian was diluted with, water/ethanol solution (25% ethanol) to 100 μg/ml and analyzed by reverse HPLC using an Eclipsed XDB C18 4.6×250 mm column with a mobile phase of water/acetonitrile/potassium dihydrogen phosphate/SDS 550 ml/450 ml/3.4 g/1.7 g at a flow of 1 ml/minute. This analysis reveals 40 peaks with 6 dominant peaks that make up ˜95% of the UV detectable components of the total mixture. The peaks are at 13.5(A), 14(B), 15(C), 17(D), 20.5(E) and 24(F) minutes. Peak F is berberine, the major constituent of huanglian. Similar to published studies²¹, it was found that berberine was ˜50% of the HPLC detectable components or approximately 25% overall. Many traditional Chinese botanicals are contaminated with arsenic. Since arsenic could not be detected by our UV monitor, the huanglian extract was tested for arsenic by atomic absorption. This method revealed only 0.1 to 0.2 μg of arsenic in each gram of solid extract. This amount of arsenic is considerably below levels that would be considered to have any biologic effect on these cells.

### EXAMPLE 8

### Metabolic Studies

Since oral drugs are metabolised by a “first-pass” through the liver, we also elected to determine whether huanglian would be metabolised in vitro by human liver S9 microsomes (Gentest, MA). This assay creates metabolites of a drug that are equivalent to those formed on a “first-pass” through the liver. For these studies, the huanglian extract, at a final concentration of 3 μ/ml, was exposed to human liver S9 microsomes at 1 mg/ml with NADPH. The microsomal extracts obtained in this assay were then analyzed by HPLC and UV spectroscopy. These results indicate that the 7 major peaks remain essentially unchanged despite the treatment with the liver microsomes. only peak #5, with a retention time of 8 minutes under these conditions; showed any significant change (loss of 15% in 3 hours), whereas the major berberine peak (#7) decreased only slightly after treatment with the liver microsomes.

### EXAMPLE 9

### Inhibition of Cell Growth by Metabolites of Huanglian and Berberine

Experiments were designed to determine whether the metabolites of huanglian would be effective in suppressing growth of tumor cells. It would be important to know whether these metabolites retain the “active components” of huanglian and suppress tumor growth to the same degree as the non-metabolised huanglian extract. For these studies the huanglian metabolites that were formed by the human liver S9 microsomes after 3 hours of exposure were utilized. The supernatant from this reaction was added directly to the culture medium in the presence of MKN-74 human gastric cancer cells. As shown in FIG. 10, with a final concentration of huanglian metabolites at 3 μg/ml (HL3ug/ml, treat-3hrs), the growth of the MKN-74 cells (as determined by the MTT assay) was suppressed to a degree that was equivalent to the non-metabolised parent compound (HL3ug/ml, treat-0hrs), when compared to the untreated control.

Since berberine constitutes the major fraction of huanglian (25%), a 10 μg/ml solution of berberine was tested to determine the degree to which it inhibited proliferation of the MKN-74 cells. Under the conditions tested, the berberine was not able to inhibit cell proliferation to the same degree as either the huanglian or its metabolites (data not shown). This supports the hypothesis that the anti-tumor effects observed in vitro are dependent on the mixture of the various components present in the huanglian extract.

### EXAMPLE 10

### Clinical Study

A. Preparation of Huanglian Extract and Testing of Purity

The material for this study will be prepared by Phoenix Laboratories, Hicksville, N.Y. under contract from Memorial Sloan-Kettering Cancer Center. 50 kg. of a “new” batch of huanglian that has an almost identical HPLC profile with identical biological activity (see above) was obtained. It is anticipated that this lot of material should provide sufficient to complete this study, and for purposes of this program will avoid issues of internal chemical or biologic variability. This batch will be provided to Phoenix who will prepare huanglian as described as follows: the root to be cut into small pieces, added to distilled water in the ratio of 30 grams to 1 liter and boiled for 3 hours with occasional stirring. The resulting volume of 250 ml to be filtered through cheese cloth and boiled for a further hour to produce 7-8 ml of thick slurry weighing just under 8 grams. After two days in a vacuum dry concentrator, the final weight of dry powder will be close to 6.00 grams, a 20% extraction. This method is amended from the method described under “preliminary data”. However, studies have been conducted that demonstrate that the biological activity is not different between the two methods of preparation. A pharmaceutical Certificate of Analysis to be provided by Phoenix will specify quality assurance as described below.

| Appearance | Brown granular powder |
|---|---|
| Solubility | Freely soluble in water with |
| | slight turbidity in 20 mg/ml |
| | of water. |
| Ingredient assay: | |
| Berberine (by HPLC as | 25% ± 5% total weight |
| described above) | |
| Identification: | |
| HPLC fingerprint (by HPLC as | There should be major peaks |
| described above) | at 13.5(A), 14(B), 15(C), |
| | 17(D), 20.5(E) and 24(F) |
| | minutes |
| Purity: (analyses to be | |
| conducted by Huffman | |
| Laboratories, Golden, CO). | |
| Heavy metals | 30 ppm maximum |
| Arsenic | 2 ppm maximum |

Huanglian will be prepared as capsules to be taken qid not within one hour of food. Capsules will initially be 250 mg but this will be doubled to 500 mg if doses escalate sufficiently to necessitate the taking of large numbers of capsules. The advantage of capsules over the traditional method of taking huanglian as a tea is that the taste of huanglian is extremely bitter.

B. A Phase I Study of the Chinese Herb Huanglian (Coptis Chinesis) in Patients with Advanced Solid Tumors

In the current protocol entitled “A Phase I Study of the Chinese Herb Huanglian (Coptis chinesis) in Patients with Advanced Solid Tumors,” patients will receive escalating doses of the aqueous extract of the Chinese herb huanglian in order to determine the maximally tolerated dose, toxicities, and to obtain preliminary information about its activity in patients with a variety of solid tumors.

Patients will receive capsules of the powdered extract of the huanglian root, which they will take by mouth 4 times a day (qid). Treatment with huanglian will continue every day without breaks until either, progression of disease, or toxicities are noted.

Endpoints which will be continuously assessed during the study are both classical toxicities (per NCI Common Toxicity Criteria, CTC Version 2.0, http://ctep.info.nih.gov), as well as surrogate markers of activity which include pharmacokinetic, biologic, and molecular laboratory determinations.

The aims of the study are as follows:

1. To conduct a phase I clinical trial with huanglian in patients with advanced solid tumors.

2. To determine the optimal dose of huanglian that is below or equal to a maximum tolerated dose and that optimises pharmacologic (i.e. plasma metabolites of huanglian), molecular (i.e. suppression of cyclin B1), and biologic (i.e. ex vivo cytotoxicity assays) parameters.

3. To pilot a methodology for developing cancer drugs from whole herbal extracts.

4. To obtain preliminary data on therapeutic activity of huanglian.

5. To utilize the results from the phase I studies to design a phase II clinical trial in gastric cancer.

Pharmacokinetic studies (PK). Bloods for pharmacokinetic studies (PK) will be performed on days 1, 2, 8, 15 and 29 of treatment. Repeated bloods for PK will be drawn on day #1 only, before the daily dose (time=0), 1 hour after, and 2,4, 6, 8 and 24 hours after the first dose of huanglian. On all other days, a single PK blood sample will be drawn, to measure steady state levels of the drug. For these studies 10 ml of heparinized blood (green stopper vacutainer tube) will be collected. Plasma samples (PK) will be sent for pharmacokinetic measurements of huanglian peaks.

Early Results

One patient entered at each of the first two dose levels

Dose level 1: 1.0 gram

Dose level 2: 1.5 gram

No clinical toxicity

Berberine detectable: steady state levels on day #15: 6 ng/ml at dose level 1 and 24 ng/ml at dose level 2.

No biologic effect.

See also FIGS. 13 and 14.

### EXAMPLE 11

Huanglian is a botanical agent prepared as a tea from the roots of Coptis chinensis. In traditional Chinese medicine it has been used to treat inflammatory conditions ranging from gastroenteritis to acute febrile illnesses with no reported toxicity. Huanglian was tested for activity against cancer. It was found that huanglian potently inhibits the growth of a number cancer cells in vitro in a dose-dependent manner, with maximal inhibition at low micromolar concentrations. MCF7 and MDA468 breast cancer lines were particularly sensitive to huanglian. The activity of huanglian was greater than an equivalent concentration of its major component, berberine, suggesting that several components contribute to its anticancer effect. It was therefore decided to take whole huanglian to human trial, as a novel departure from the conventional method of selecting and testing a single active compound. In addition to single agent activity against breast cancer cell lines, huanglian was also shown to enhance the effect of paclitaxel, supporting the future development of huanglian in combination with paclitaxel for the treatment of patients with metastatic breast cancer.

### Research Aims and Results

The overall goal for this grant is to develop new therapeutic approaches in the treatment of patients with metastatic breast cancer based utilizing the Chinese botanical huanglian. The specific aims are to:

1) To conduct a phase I clinical trial of huanglian with both toxicity and efficacy endpoints.

2) Based on the results of the phase I clinical trial of single agent huanglian, conduct a phase I/II clinical trial of huanglian in combination with paclitaxel in the treatment of patients with metastatic breast cancer.

1. “A Phase I Study of the Chinese Herb Huanglian (Coptis chinesis) in Patients with Advanced Solid Tumors” (MSKCC Protocol Number 00-061A(6)) has now been open for the past year. The purpose of this study is to determine the optimal dose of huanglian for future phase II trials. Patients with advanced solid tumors who have failed all conventional therapy or for which there is no conventional therapy are eligible for this study. Twenty-one patients have been registered to this study. One patient elected to withdraw consent after study registration and never received huanglian.

Defining the MTD: The initial study design utilized a rapid dose escalation schedule of 1 patient/level and the huanglian dose was to be increased by 50% in successive cohorts. The starting dose of huanglian was 1 gm/day or one capsule (250 mg/tablet), p.o., 4×/day. At dose level 3 (2.25 gm/day), one additional patient was added since the first patient developed progression of disease (POD) before completing her assessment for toxicity. Using this study design, a dose of 3.5 gm/day or 14 capsules in 4 divided doses was escalated to. At a dose of 5.25 gm/day (21 capsules/day), one patient developed grade 3 diarrhea (DLT) and the cohort was expanded to 6 patients with no further DLTs noted. However, because of this toxicity, the study design now changed to a classic dose escalation schema of 3 to 6 patients/dose level and a 25% dose escalation in all successive cohorts. Utilizing this approach, the dose was escalated to 6.56 gm/day (26 capsules/day) in 3 patients without DLT. In the next cohort of 8.25 gm/day (33 capsules/day), again 1 patient was observed with grade 3 diarrhea. This cohort has recently been expanded to 6 patients and therapy is still ongoing. These results are summarized in the table below, which also indicates stable disease as best response in several patients on the study. Each of these patients had been progressing under observation or on therapy before entering the clinical trial with huanglian.

| | Pt. | Dose | Pill | | |
|---|---|---|---|---|---|
| Cohort | # | (gm/day) | # | Toxicity | Best Response |
| 1 | 1 | 1 | 4 | 0 | Stable (colon): 6.4 |
| | | | | | months |
| 2 | 1 | 1.5 | 6 | 0 | Stable (neuro): 12 |
| | | | | | months |
| 3 | 2 | 2.25 | 9 | 0 (1 NE) | None |
| 4 | 1 | 3.5 | 14 | 0 | None |
| 5 | 6 | 5.25 | 21 | 1: gr. 3 | Stable (sarcoma): |
| | | | | diarrhea | 6.5⁺ months |
| 6 | 3 | 6.56 | 26 | 0 | Stable (renal): |
| | | | | | 5.2⁺ months |
| 7 | 1 | 8.2 | 33 | 1: gr 3 | Too early |
| | | | | diarrhea | |

Pharmacokinetic (PK) and surrogate marker studies: Since berberine represents approximately 50% of the herb, PK measurements were being conducted of berberine by HPLC. However, only very low levels of berberine (<50 ng/ml) have been detected in the plasma of any patient on the study. Also being tested is plasma collected from patients on day #15 of therapy to determine if the patient's own plasma will inhibit cancer cell growth of the gastric cancer cell line MKN-74 ex vivo, as determined by SRB assays. For these studies, the patient's plasma is concentrated and the MKN-74 cells are exposed to the patient's own plasma for either 2 (II) or 4 (IV) days before the tumor cells are assayed for growth inhibition. As shown for the patient treated in cohort 3 (see graph below), the patient's own pretreatment plasma (day 0) was capable of inhibiting MKN-74 cell growth, even though the patient had been off all chemotherapy for at least 4 weeks. Three separate samples were then drawn on day 15 of treatment at 8:30 AM, 9:30 AM, and 12:30 PM. As shown, when the MKN-74 cells were exposed ex vivo to the patient's plasma for 2 (II) or 4 (IV) days, there was inhibition of cell growth in all three samples when compared to the patient's pretreatment plasma for both the 2 and the 4 day assay. The last lanes show the positive control of media “spiked” with huanglian (HL) at a concentration of 20 μg/ml and exposed to MKN-74 cells for both 2 and 4 days. This degree of growth suppression confirms the activity of the herb from the capsules.

Despite these promising results, this pattern of growth inhibition, which has varied from 0 to 60% in the patients on the study, has been highly variable from patient to patient. It has also shown no correlation to dose or to treatment response. Nevertheless, it does suggest some degree of biologic activity from the preparation under investigation. Since huanglian has also been shown to inhibit cyclin B1 protein expression in the same cell line, we have also performed western blots for cyclin B1 expression in the MKN-74 cells in these same samples. For these studies the cyclin B1 was either not detectable at baseline and was considered non-informative or showed no suppression in this ex vivo assay.

The plan is to complete this phase I study of huanglian, so as to define the MTD and then test this in phase II clinical trials. Especially encouraging is the stable disease in at least 3 patients with advanced cancers (renal, sarcoma, and neuroendocrine tumors) who were progressing either under observation or on chemotherapy. These patients had no other treatment options at the point of study entry.

It is conceivable that for huanglian we may not be able to define an MTD with standard grade 3 and 4 dose-limiting toxicity. Instead the highest non-toxic dose may be determined by the number of capsules consumed at any one time. In fact, if escalated to the next cohort, without a second DLT at the current dose level, patients will be taking 41 capsules/day, divided in 4 doses. In its current formulation, this may represent a pill count which will exceed that which a patient can take at any given time. The SRB assays on the MKN-74 cells from the patient's own plasma indicate a pharmacodynamic effect, even at doses of huanglian as low as 1.5 gm/day (cohort 2). Though this has not correlated to treatment response, and it has not achieved our target of 50% inhibition of cell growth in all patients, it does suggest that plasma levels of huanglian that can induce some degree of a biologic effect are being achieved. Without tumor biopsies to indicate biologic effects in the tumor, it is impossible to know what in fact is taking place in the tumor. However, it does suggest that plasma levels of some component of huanglian (other than berberine, which is non-measurable by HPLC) which is capable of inhibiting tumor growth ex vivo are being obtained. Thus, in the case of huanglian it may be possible to select a dose that is considerably below the MTD for future clinical development.

2. The next step in drug development will be to take the highest non-toxic dose or that dose which gives us our greatest biologic effect ex vivo, and use this dose to conduct a phase I trial of huanglian and paclitaxel in 10 to 20 patients with advanced solid tumors. This study is based on preclinical studies indicating that huanglian enhances the effect of paclitaxel in vitro. There is no evidence of any antagonism between huanglian and paclitaxel in vitro. Once the MTD of huanglian and paclitaxel is determined in this planned phase I trial, a phase II trial with this combination will then be conducted in 20 to 30 patients with metastatic breast cancer. This clinical trial will be supported by Department of Defense (DOD) grant awarded from the Army Breast Cancer Research Fund. Based on the current phase I trial of single agent huanglian, no serious or adverse events greater than that which is achieved with single agent paclitaxel alone are anticipated.

### EXAMPLE 12

### Phase I Study of the Chinese Herb Huanglian (HL): Evidence of Biologic and Clinical Activity

HL is an herbal tea used in traditional Chinese medicine. It has been reported that HL, obtained from the dry precipitate of the water soluble, heat extractable fraction, inhibits tumor cell growth by suppressing cyclin B1 protein, inhibiting cdc2 kinase and inducing a G2 arrest. (Mol. Pharm.58:1287, 00). By HPLC, 50% of this aqueous fraction consists of berberine (B). A phase I trial of HL was conducted utilizing a rapid dose escalation design (1 patient/cohort). The starting dose was 1 gr or 1 capsule (250 mg), p.o, 4×/day, taken daily until disease progression. In the absence of toxicity, an active dose for phase II studies based on biologic markers of response was sought. For these assays, plasma was obtained on days 0, 1 and 14 to measure berberine by HPLC, and to treat a reference human gastric cancer cell line with the patient's own plasma in vitro for inhibition of cell growth by sulforhodamine B (SRB) assay and for suppression of cyclin B1 by western blot. The biologic end-point was empirically defined as a positive response on 2 of the following 3 assays: ≧50% inhibition of cell growth by SRB, suppression of cyclin B1 by western or plasma B levels ≧0.75 μg/ml. To date 7 patients have entered the study. Median age is 53 (range: 19-51), KPS 80(70-90), prior regimens 5(3-14). Tumor types: colon (2), hepatoma (2), breast, sarcoma, carcinoid. 4 cohorts (1.0, 1.5, 2.25, and 3.5 gr/day) without any toxicity have been completed. In cohort 5 (5.25 gr/day), there has been 1 DLT (grade 3 diarrhea) and the cohort is being expanded. Biologic activity, consisting of 40%-60% inhibition, on either the SRB or cyclin in B1 assay, has been seen at all dose levels ≧1.5 gr/day. Plasma B is barely detectable (0-0.024 μg/ml). Disease stabilization has been seen in 3 patients (2 too early, 1 inevaluable) and the median duration on study is 3.1 months (range: 1-6.8+). In conclusion, HL is well tolerated and there is evidence of biologic and clinical activity, independent of plasma B levels. Since neither a standard toxicity end-point nor a biologic end-point (2 of 3 biologic responses in any one patient) has been reached, this study continues to accrue patients to define a phase II dose.

### REFERENCES

1. Zhang L., Yang L, Zheng X. A study of heliocobacterium pylori and prevention and treatment of chronic atrophic gastritis. J Tradit Chin Med 17: 3-9, 1997.

2. Yang L Q, Singh M, Yap E H, Ng G C, Xu H X, and Sim K Y. In vitro response of Blastocystis hominis against traditional Chinese medicine. J. Ethnopharmacol 55: 35-42, 1996.

3. Franzblau S G, and Cross C. Comparative in vitro antimicrobial activity of Chinese medicinal herbs. J. Ethnopharmacol 15: 279-288, 1986.

4. Song L C Chen K Z Zhu J Y, The effect of Coptis chinensis on lipid peroxidation and antioxidases activity in rats, Chung Kuo Chung Hsi I Chieh Ho Tsa Chih (1992 July) 12(7):421-3, 390

5. Umeda M Amagaya S Ogihara Y, Effects of certain herbal medicines on the biotransformation of arachidonic acid: a new pharmacological testing method using serum. J Ethnopharmacol (1988 May-June) 23(l):91-8

6. Wang G D Zhang Y M Xiong X Y, Clinical and experimental study of burns treated locally with Chinese herbs, Chung Hsi I Chieh Ho Tsa Chih (1991 December) 11(12):727-9, 709

7. Huang W M Yan J Xu J, Clinical and experimental study on inhibitory effect of sanhuang mixture on platelet aggregation, Chung Kuo Chung Hsi I Chieh Ho Tsa Chih (1995 August) 15(8):465-7

8. Kobayashi Y, Yamashita Y, Fuji N, Takaboshi K, Kawakami T, Kamwamura M, Mizukami T, and Nakano H. Inhibitors of DNA topoisomerase I and II isolated from the Coptis rhizomes. Planta Med 61: 414-418, 1995.

9. Yamashita Y, Fujii N, and Nakano H. Identification of novel topoisomerase I inhibitors. Proc. Amer. Assoc. Cancer Res. 35: 707, 1994.

10. Fang X P, Wang T Z, Shang H, Shuai H, Li D, and Xie C K. Quantitative determination of 5 alkaloids in plants of coptis from china. Chung Kuo Yao Tsa chih 14: 33-35, 1989.

11. Chi C W, Chang Y F, Cjao T W, Chiang S H, P'eng F K, Lui W Y, and Liu T Y. Flowcytometric analysis of the effect of berberine on the expression of glucocorticoid receptors in human hepatoma HepG2 cells. Life Sci 54: 2099-2107, 1994.

12. Fukutake M, Yokota S, Kawamura H, Iizuka a, Amagaya S, Fukuda K, and Komatsu Y. Inhibitory effect of Coptidis Rhizoma and Scutellariae Radix on azoxymethane-induced aberrant crypt foci formation in rat colon. Bio. Pharm Bull 21: 814-817, 1998.

13. Qin C L, Liu J Y, and Cheng Z M. Pharmacological studies on the effect of huanglian decoction on experimental gastric lesions in rats and antiemetic in pigeons. Chung Kuo Chung Yao Tsa Chih 19: 427-430, 1994

14. Yeung C Y, Lee F T, and Wong H N. Effect of a popular Chinese herb on neonatal bilirubin protein binding. Biol Neonate 58: 98-103, 1990.

15. Chan E, Displacement of bilirubin from albumin by berberine. Biol Neonate (1993) 63(4):201-8

16. Marin-Neto J A, Maciel B C, Seecches A L, Gallo L. Cardiovascular effects of berberine in patients with severe congestive heart failure. Clin Cardiol 11: 253-260, 1988.

17. Zeng X, and Zeng X. Relationship between the clinical effect of berberine on severe congestive heart failure and its concentration in plasma studied by HPLC. Biomed Chromatogr 13: 442-444, 1999.

18. Gao X S, Absorptive capacity of upper gastrointestinal tract with Chinese herbal medicine, Chung Kuo Chung Hsi I Chieh Ho Tsa Chih (1993 July) 13(7):433-5, 390

19. Ozaki Y, Suzuki H, and Satake M. Comparative studies on concentration of berberine in plasma after oral administration of coptidis rhizoma extract, its cultured cells extract and combined use of these extracts and glycyrrhizai radix extract in rats. Yakugaku Zasshi 113: 63-69, 1993.

20. Nurse P. Universal control mechanism regulating onset of M phase nature (Lond) 344: 503-508, 1990.

21. Chuang W-C, Young D-S, Liu LK, and Sheu S-J. Liquid chromatography-electrospray mass spectrometric analysis of coptidis Rhizoma. J. Chromatog 755L 19-26, 1996.

22. Chen C M, Chang H C. Determination of berberine in plasma, urine, and bile by HPLC. J. Chromatogr. 117-123, 1995.

23. Zhang H, Want T, Fang X, Shuai H, and Xie C. Plants of coptis from Tibet and Yunnan. Chung Kuo Chung Yao Tsa Chih China Journal of Chinese Materia Medica. 15: 326-328, 1990.

24. Walker D L, Reid J M, Ames M M. Preclinical pharmacology of bizelesin, a potent bifunctional analog of the DNA-binding antibiotic CC-1065. Cancer Chemother Pharmacol 34: 317-322, 1994.

25. Response Evaluation Criteria in Solid Tumors (RECIST criteria), Revised version of the WHO criteria published in the WHO Handbook for reporting results of cancer treatment (Geneva, 1979), revised June 1999, personal communication, CTEP, NCI.

What is claimed is:

1. A method for screening a mixture of compounds for activity comprising steps of:
a. contacting the mixture with a system which mimics an organ capable of metabolizing the mixture in an appropriate time to generate metabolites; and
b. determining the activity of the generated metabolites.

2. A method for quantitating a mixture of compounds comprising steps of:
a. contacting the mixture with a system which mimics an organ capable of metabolizing the mixture in an appropriate time to generate metabolites; and
b. determining the activity of the generated metabolites.

3. A method for identification of an active metabolite of a mixture of compounds comprising steps of:
a. contacting the mixture with a system which mimics an organ capable of metabolizing the mixture in an appropriate time to generate metabolites; and
b. determining the activity of the generated metabolites.

4. The method of claim 1, 2 or 3, wherein the organ is a liver.

5. The method of claim 4, wherein the system is human liver microsomes.

6. A method for screening a mixture of compounds for activity comprising steps of:
a. administering the mixture to a subject capable of metabolizing the mixture; and
b. taking bodily fluid which contains metabolites of the mixture from the subject, to determine the activity of the metabolite.

7. A method for quantitating an herbal extract comprising steps of:
a. administering the mixture to a subject capable of metabolizing the mixture; and
b. taking bodily fluid which contains metabolites of the mixture from the subject, to determine the activity of the metabolite.

8. A method for identification of the active metabolite of a mixture of compounds comprising steps of:
a. administering the mixture to a subject capable of metabolizing the mixture; and
b. taking bodily fluid which contains metabolites of the mixture from the subject, to determine the activity of the metabolite.

9. The method of claim 1, 2, 3, 6, 7, or 8, wherein the activity of the metabolites is determined by in vitro assay.

10. The method of claim 9, wherein the assay is determined by the inhibition of cell growth.

11. The method of claim 10, wherein the cells are cancerous cells.

12. The method of claim 1, 2, 3, 6, 7, or 8, wherein the activity of the metabolites is determined by inhibition of cyclin B1 activity.

13. The method of claim 12, wherein the inhibition is at least 50%.

14. The method of claim 1, 2, 3, 6, 7, or 8, wherein the mixture is from a natural product.

15. The method of claim 14, wherein the natural product is an herb.

16. The active metabolite identified by the method of claim 3 or 8.

17. A pharmaceutical composition comprising an effective amount of the metabolite of claim 16 and a pharmaceutically acceptable carrier.

18. A method of producing a fingerprint of an extract of a natural product comprising steps of:
a. contacting the extract with a system which mimics an organ capable of metabolizing the extract in an appropriate time to generate metabolites; and
b. determining the identity and amount of the metabolites generated, thereby generating a fingerprint of the extract.

19. The method of claim 18, wherein the organ is a liver.

20. The method of claim 19, wherein the system is human liver microsomes.

21. A method of producing a fingerprint of an extract of a natural product comprising steps of:
a. administering the extract to a subject capable of metabolizing the extract; and
b. determining the metabolites generated, thereby generalizing a fingerprint of the extract.

22. The method of claim 13 or 16, wherein the natural product is an herb.

23. The fingerprint produced by claim 18 or 21.

24. A method to determine the batch-to-batch variation of an extract from a natural product comprising comparison of the fingerprint of claim 23 of different batches.

25. A method to assay for formulation variation of an extract from a natural product comprising comparison of the fingerprint of claim 23 of different batches.

26. A method to assay for dose variation of an extract from a natural product comprising comparison of the fingerprint of claim 23 of different batches.

27. A method for identification of induced compounds in a subject comprising steps of:
a. administering a mixture of compounds to the subject; and
b. extracting bodily fluid from the subject to determine the generation of induced compound; and
c. identifying said induced compound.

28. The method of claim 27, wherein the mixture is an extract from a natural product.

29. The induced compounds identified by claim 27.

30. The method of claim 6, 7, 8, 21, or 27, wherein the subject is a human.

31. A method for treating cancer in a subject comprising administering to the subject an effective amount of coptis chinesis extract.

32. The method of claim 31, wherein the cancer is a solid tumor.

33. A method for treating cancer in a subject comprising administering to the subject an effective amount of coptis chinesis extract and a therapeutic agent.

34. The method of claim 33, further comprising a protein kinase C inhibitor.

35. The method of claim 33, wherein the therapeutic agent is a microtubule-destabilizing agent.

36. The method of claim 33, wherein the treating of the coptis chinesis extract and the therapeutic agent is performed in a sequential manner.

37. The method of claim 36, wherein the subject is treated with coptis chinesis extract first, then a therapeutic agent.

38. The method of claim 37, wherein the therapeutic agent is a microtubule-destabilizing agent.

39. The method of claim 35 or 38, wherein the microtubule-destabilizing agent is a taxol or taxol-like compound.

40. An anti-tumor composition comprising an effective amount of coptis chinesis extract.
